(19) United States
(12) Patent Application Publication
(10) Pub. No.: US 2026/0315031 A1
(43) Pub. Date: Oct. 15, 2026

(54) ULTRASOUND APPARATUS INTEGRATING DIAGNOSIS AND THERAPY

(71) Applicant: Guangzhou Institute of Cancer Research, the Affiliated Cancer Hospital, Guangzhou Medical University, Guangzhou (CN)

(72) Inventors: Chunyue LUO, Guangzhou (CN); Zuojia LI, Guangzhou (CN); Yutong LIU, Guangzhou (CN); Jinghan HU, Guangzhou (CN)

(21) Appl. No.: 19/464,016

(22) Filed: Jan. 29, 2026

Related U.S. Application Data
(63) Continuation of application No. PCT/CN2025/088505, filed on Apr. 11, 2025.

(30) Foreign Application Priority Data
Apr. 8, 2025 (CN) 202510431832.7

(51) Int. Cl. A61B 8/00; A61N 7/00

(52) U.S. Cl. CPC A61B 8/461; A61B 8/4405; A61B 8/4444; A61B 8/54; A61N 7/00; A61N 2007/0052

(57) ABSTRACT

The present invention discloses an ultrasound apparatus integrating diagnosis and therapy in the field of ultrasonic detection technology, including a B-mode ultrasound machine, where a controller is disposed on the B-mode ultrasound machine, a first motor, a support disc, a second motor, a support arm, a bidirectional motor, an extension arm, and a display are disposed at a top of the B-mode ultrasound machine, a plurality of electric push rods are unidirectionally and fixedly connected to a side wall of a top of the extension arm, each of the electric push rods is ball-jointed to the display, a camera and a laser radar are fixedly connected to the display, and the first motor, the second motor, the bidirectional motor, the electric push rod, the display, the camera, and the laser radar are all in signal connection with the controller.

### CROSS-REFERENCE TO RELATED APPLICATIONS

The present application is a continuation of International Application No. PCT/CN2025/088505, filed on April 11, 2025, which claims priority to Chinese patent application No. 202510431832.7, titled "ULTRASOUND APPARATUS INTEGRATING DIAGNOSIS AND THERAPY", filed on 8 April 2025, the entire contents of which are incorporated herein by reference.

### FIELD OF TECHNOLOGY

The present invention pertains to the field of ultrasonic detection technology, and specifically to an ultrasound apparatus integrating diagnosis and therapy.

### BACKGROUND

An ultrasound apparatus integrating diagnosis and therapy mainly relies on the physical properties of ultrasound, namely the reflection, the refraction, and the absorption of ultrasound in human tissues. These properties enable ultrasound to penetrate human tissues, to form images of internal structures, and to monitor changes of the internal structures in real time.

Existing B-mode ultrasound machines mostly adopt a split-type design, in which a handheld ultrasound probe is connected to a B-mode ultrasound machine and a display via cables. This design often results in inconsistency between the detection position of medical personnel and the display orientation angle of the B-mode ultrasound machine, and interferes with the efficiency of medical personnel in observing the B-mode ultrasound display while moving during detection to locate lesion images.

Moreover, since medical personnel are required to wear medical gloves during B-mode ultrasound examination, it is very inconvenient to hold the ultrasound probe or to adjust the detection parameters of the B-mode ultrasound machine. This inconvenience may cause hygiene issues during detection, thereby affecting the efficiency of B-mode ultrasound detection, and reducing the accuracy of ultrasound examination and diagnosis.

Therefore, it is necessary to provide an ultrasound apparatus integrating diagnosis and therapy, which can automatically adjust the three-dimensional angle of the display screen of the B-mode ultrasound machine following the viewpoint of medical personnel, and allow medical personnel to independently adjust the angle of the display screen of the B-mode ultrasound machine and the detection parameters of the B-mode ultrasound machine through non-contact means.

### SUMMARY

In order to resolve the above problems, an objective of the present invention to provide an ultrasound apparatus integrating diagnosis and therapy, which recognizes and tracks the face of an operator through a self-driven display support arm and extension arm, in combination with a camera and a laser radar on the display, always orients toward the face of the operator, and further combines non-contact command methods to reduce the working difficulty during ultrasonic detection, enhancing the accuracy of detection and diagnosis by the operator, and avoiding diagnostic errors caused by difficulty in observing the display.

In order to achieve the above objective, the technical solution of the present invention is as follows: The ultrasound apparatus integrating diagnosis and therapy includes a B-mode ultrasound machine, a controller is disposed on the B-mode ultrasound machine, a first motor oriented vertically upward is fixedly connected to the top of the B-mode ultrasound machine, a support disc is rotatably connected to a top wall of the B-mode ultrasound machine, the support disc is coaxially and fixedly connected to an output shaft of the first motor, a horizontal second motor is fixedly connected to the support disc, a support arm is pivotally connected to a center of the support disc, an output shaft of the second motor is coaxially and fixedly connected to a pivot point at a bottom end of the support arm, a bidirectional motor is horizontally and fixedly connected to a middle portion of the support arm, a motor gear is coaxially and fixedly connected to each of output ends on two sides of the bidirectional motor, an extension arm is pivotally connected to a top end of the support arm, an arm gear is coaxially and fixedly connected to each of two sides of a pivot point at a bottom end of the extension arm, a transmission chain is sleeved between the motor gear and the corresponding arm gear, a plurality of electric push rods are unidirectionally and fixedly connected to a side wall of a top of the extension arm, a display is ball-jointed to a top end of the extension arm, each of the electric push rods is ball-jointed to the display, and a camera and a laser radar are fixedly connected to the display.

The first motor, the second motor, the bidirectional motor, the electric push rod, the display, the camera, and the laser radar are all in signal connection with the controller.

The principle of the basic solution is as follows: After the operator starts the apparatus, the camera and the laser radar begin to operate, and capture and track the facial position of the operator in real time. The controller processes the image data, drives the motors and the electric push rods to adjust the position and the angle of the display, and makes the display always oriented toward the face of the operator.

The beneficial effects of the basic solution are as follows:
1. The apparatus achieves automatic positioning and tracking of the display through the integrated camera, the laser radar, and the motor drive system. This means that the operator does not need to frequently manually adjust the angle and the position of the display, thereby greatly reducing complicated steps during operation and improving overall operational convenience. The addition of the voice recognition system enables the operator to control various functions of the apparatus through simple voice commands. This not only simplifies the operation process, but also reduces possible errors caused by manual operation, further improving work efficiency.
2. Through the facial tracking technology of the built-in system in the controller, the display can always be maintained within the optimal viewing angle range of the operator. This not only ensures that the operator can clearly observe the B-mode ultrasound images, but also avoids diagnostic errors caused by poor viewing angles, thereby improving diagnostic accuracy. The built-in sensor system of the apparatus can monitor the facial position and the movements of the operator in real time, and once deviation from the optimal viewing angle is detected, the system immediately makes adjustments. This real-time feedback mechanism ensures that the operator can always obtain the most accurate diagnostic information.
3. In the context of frequent public health events such as pandemics, non-contact command methods are particularly important. The apparatus achieves non-contact operation control through the facial tracking technology and the voice recognition system, and effectively avoids the risk of cross-infection caused by contact transmission, improving the operational safety.
4. During facial tracking, the first motor rotates the support disc to adjust the overall direction of the support arm and the display, the second motor controls the raising and the lowering of the support arm, the bidirectional motor drives the raising and the lowering of the extension arm through the transmission chain and the arm gears, and the electric push rods on the extension arm push the ball-jointed display at the tail end of the extension arm to change orientation, thereby achieving smooth and rapid adjustment of the display in the three-dimensional angle direction, facilitating tracking of the facial orientation of the operator, and ensuring that the display always faces the operator directly.

Further, one side of the B-mode ultrasound machine is fixedly connected to one end of a cable, the other end of the cable is fixedly connected to a handheld ultrasound probe, a remote controller is fixedly connected to a tail of the ultrasound probe, a plurality of buttons and knobs are disposed on the remote controller, and the remote controller is in signal connection with the controller.

The beneficial effects of the basic solution are as follows:
1. The cable helps the operator to freely move the ultrasound probe to obtain a wider and deeper scanning area. The handheld ultrasound probe not only improves the operational flexibility, but also helps the operator to more accurately locate lesions or abnormal areas, thereby improving the diagnostic precision.
2. The remote controller allows the operator to perform real-time adjustments to various functions of the B-mode ultrasound machine while holding the handheld ultrasound probe. This design reduces the need for the operator to frequently switch operation positions, and helps the operator to more focus on the scanning process, improving the operational convenience and efficiency.
3. Through the buttons and the knobs on the remote controller, the operator can instantly adjust scanning parameters of the ultrasound probe, such as depth, gain, and frequency. This instant adjustment capability helps the operator to quickly capture key information, reduces the possibility of repeated scanning and misdiagnosis, and thereby improves diagnostic efficiency and accuracy.

Further, a real-time audio receiving device is disposed in the controller, and the real-time audio receiving device is in signal connection with the controller.

The beneficial effects of the basic solution are as follows:
1. On the basis of the original camera and laser radar, the real-time audio receiving device additionally disposed at the top of the B-mode ultrasound machine, after being in signal connection with the controller, achieves multi-modal interaction methods such as voice and facial recognition. The operator can control the apparatus through voice commands and facial orientation, further enhancing operational convenience and flexibility.
2. The real-time audio receiving device is not only used to receive voice commands from the operator, but also captures feedback sounds during the operation process, such as comments or questions from the operator about the images. By analyzing these sound information through intelligent algorithms, the apparatus can automatically adjust display parameters, enlarge key regions, or provide additional diagnostic suggestions, thereby enhancing interactivity during the operation process.
3. The real-time audio receiving device allows the operator to make voice annotations during the examination process, and these annotations can be automatically synchronized to the B-mode ultrasound images or diagnostic reports. This not only helps the operator to quickly record key information, but also provides valuable references in subsequent analysis, thereby improving diagnostic precision and efficiency.
4. The intelligent voice recognition system equipped with the real-time audio receiving device can not only recognize the voice commands of the operator, but also perform identity verification. This means that only authorized operators can control the apparatus, effectively preventing unauthorized access and operation, and enhancing operational security.

Further, a plurality of casters are fixedly connected to a bottom wall of the B-mode ultrasound machine.

The beneficial effects of the basic solution are as follows: The plurality of casters fixedly connected to the bottom of the B-mode ultrasound machine provide the B-mode ultrasound machine with flexible mobility, facilitating movement of the B-mode ultrasound machine in various situations and rooms, and making it convenient for medical personnel to operate the B-mode ultrasound machine for ultrasonic examination.

Further, a light shield is pivotally connected to a top wall of the display, one end of a pull rope is pivotally connected to a top wall of the light shield, and the other end of the pull rope is pivotally connected to one side of the bottom end of the extension arm.

The beneficial effects of the basic solution are as follows:
1. The light shield pivotally connected to the top wall of the display can be freely flipped and adjusted in angle according to actual needs, effectively blocks stray light from different directions, and provides the operator with a clearer and interference-free field of view. This design helps the operator to easily control the light shield without frequently moving or adjusting the position during the examination process, and improves the operational convenience.
2. Through the pivotal connection design between the pull rope and the extension arm, when the electric push rods on the extension arm push the display to tilt downward, the height and the position of the light shield can be adaptively adjusted to meet observation requirements at different angles. The design of the pull rope makes the adjustment process more automated without requiring additional operation steps, and further enhances operational flexibility.
3. The effective use of the light shield can significantly reduce the influence of surrounding ambient light on the display, decrease issues such as image blur and reflection, and make B-mode ultrasound images clearer and more accurate. This helps the operator to capture and analyze image information more accurately, and improves diagnostic precision and reliability.
4. The design of the light shield and the pull rope enables the B-mode ultrasound machine to adapt to different examination environments and scenarios, such as changes in indoor lighting and outdoor examinations. This adaptability helps to expand the application range of the B-mode ultrasound machine, and improves device utilization.

Further, one end of a retractable light curtain is fixedly connected to each of two sides of the light shield, and the other end of the retractable light curtain is fixedly connected to a corresponding side of two sides of the display.

The beneficial effects of the basic solution are as follows:
1. The addition of the retractable light curtains significantly enhances the shading effect of the light shield. Regardless of whether the apparatus is in a strong light environment or in a diagnostic scenario requiring high concentration, the retractable light curtains can effectively block side light, and provide the operator with a clearer and interference-free field of view.
2. The design of the retractable light curtains allows free adjustment of the shading range when the light shield is raised. No matter what angle it is in, complete shading of the sides can be achieved through the retraction and extension of the retractable light curtains, thereby meeting the needs in different scenarios.

Further, the light shield and the retractable light curtains are black.

The beneficial effects of the basic solution are as follows:
1. Black is a color with extremely high shading performance, and black can absorb most of the light and reduce the reflection and transmission of light. Therefore, designing the light shield and the retractable light curtains in black further enhances their shading effect, effectively blocks interference from external light, and provides the operator with a clearer and interference-free field of view.
2. Black has low brightness, and does not cause strong visual stimulation. Therefore, using black light shields and retractable light curtains can reduce visual fatigue generated when the operator stares at the display for a long time, improving the work efficiency and comfort of the operator.

Further, a protective shell is fixedly sleeved on a periphery of each of the support disc, the support arm, and the extension arm.

The beneficial effects of the basic solution are as follows:
1. The protective shell can tightly wrap key components such as the support disc, the support arm, and the extension arm, effectively preventing them from being damaged by collision, scratching, or squeezing during use. This helps to extend the service life of the device and reduce maintenance and replacement costs. The addition of the protective shell enhances the overall rigidity and stability of the device, and makes the device more stable during placement and movement, and less prone to tipping or damage. This helps to ensure safety during operation, reducing potential accident risks.
2. The protective shell can absorb and reduce noise and vibration generated by the device during operation, providing the operator with a quieter and more comfortable working environment. This helps to reduce the fatigue of the operator, improving the work efficiency. The surface of the protective shell is usually smooth and easy to clean, and this makes daily cleaning and maintenance of the device simpler and more convenient. This helps to maintain the cleanliness and hygiene of the device, reducing the risk of cross-infection.
3. The addition of the protective shell helps the device to better adapt to different working environments. Regardless of whether the device is in humid, dusty, or other harsh conditions, the protective shell can provide effective protection for the device, ensuring normal operation of the device.

Further, a facial recognition module, a facial orientation tracking module, an eye tracking module, a voice recognition module, and a command output module are disposed in the controller;
the facial recognition module is configured to combine image data of the camera and depth data of the laser radar to record facial features of an operator and to identify a recorded operator;
the facial orientation tracking module is configured to combine the image data of the camera and the depth data of the laser radar, to use a pre-trained LVQ neural network to recognize and track a face orientation, and to output three-dimensional angle data of the display;
the eye tracking module is configured to combine the image data of the camera and the depth data of the laser radar, to use a pre-trained convolutional neural network to calculate an eye posture, to establish a gaze model, and to determine whether a line of sight of the operator is concentrated on a display screen;
the voice recognition module is configured to extract a voice feature of the operator and to identify voice text content; and
the command output module is configured to, based on the three-dimensional angle data of the display, a line-of-sight determination result, and the voice text content, output an instruction to control a three-dimensional angle change of the display, to control starting or pausing of an angle change of the display, and to adjust parameters of the B-mode ultrasound machine.

The beneficial effects of the basic solution are as follows:
1. The facial recognition module can accurately record and identify the operator, allowing for automatic adjustment of the device based on personal preferences. The facial orientation tracking module and the eye tracking module can track the facial orientation and the line of sight of the operator in real time, ensuring that the display always remains at the optimal viewing angle, which reduces the fatigue of the operator.
2. The voice recognition module helps the operator to control the device through voice commands without manual operation, greatly improving operational convenience. This is particularly important for operators working in busy or special environments.
3. Through the synergistic effect of the facial orientation tracking module and the command output module, the device can automatically adjust the angle of the display to adapt to different heights and postures of the operator, improving viewing comfort and work efficiency. The integration of the voice recognition module and the command output module helps the operator to avoid frequent manual adjustments to the device, thereby reducing interference and fatigue during operation, and improving work efficiency.

Further, the LVQ neural network in the facial orientation tracking module includes an input layer, a hidden layer, and an output layer, and is pre-trained using face images at different angles captured by the camera and the depth data obtained by the laser radar.

The beneficial effects of the basic solution are as follows:
1. The LVQ neural network combines data of face images captured by the camera and depth data acquired by the laser radar, and achieves precise tracking of the facial orientation of the operator. This fusion of multi-dimensional data improves the accuracy and stability of tracking.
2. The facial orientation tracking module can track the facial orientation of the operator in real time, and automatically adjust the angle of the display based on the tracking results. This helps the operator to maintain the optimal viewing angle without manually adjusting the display, thereby improving the operational convenience and comfort. Through intelligent tracking and automatic adjustment, interference and fatigue caused by frequent manual adjustment of the display by the operator are reduced. This helps the operator to focus more on the diagnostic process, improving the work efficiency and accuracy.

### BRIEF DESCRIPTION OF THE DRAWINGS

FIG. 1 is a front axonometric view of an ultrasound apparatus integrating diagnosis and therapy according to an embodiment of the present invention.

FIG. 2 is a rear axonometric view of the ultrasound apparatus integrating diagnosis and therapy according to an embodiment of the present invention.

FIG. 3 is a front view of the ultrasound apparatus integrating diagnosis and therapy according to an embodiment of the present invention.

FIG. 4 is a side sectional view of the ultrasound apparatus integrating diagnosis and therapy according to an embodiment of the present invention.

FIG. 5 is a schematic diagram of a controller system of the ultrasound apparatus integrating diagnosis and therapy according to an embodiment of the present invention.

### DESCRIPTION OF THE EMBODIMENTS

The following further describes the present invention in detail through specific implementations:

### Embodiment 1

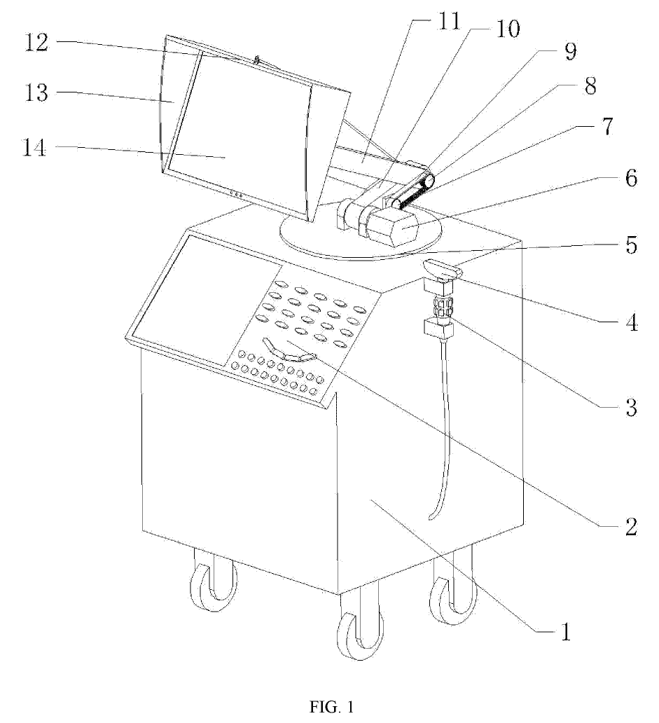

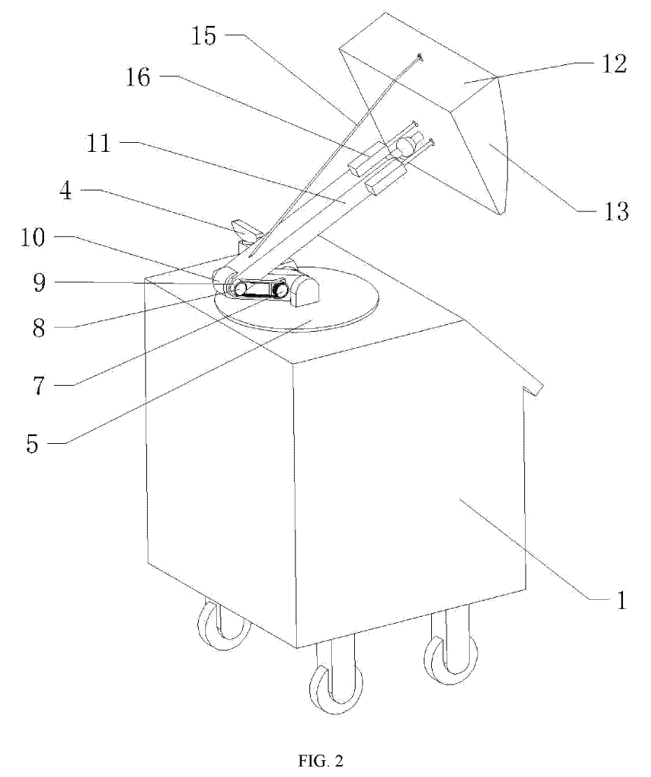

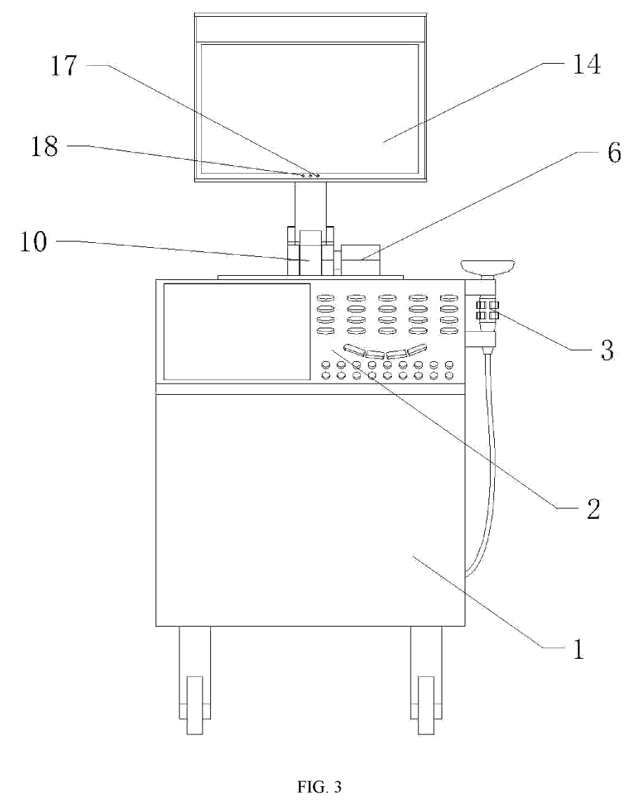

Basically, as shown in FIGS. 1, 2, 3, and 4: an ultrasound apparatus integrating diagnosis and therapy is provided, including a B-mode ultrasound machine 1. A controller 2 is installed on the B-mode ultrasound machine 1, and a first motor 19 oriented vertically upward is welded to the top of the B-mode ultrasound machine 1. A support disc 5 is rotatably connected to the top of the B-mode ultrasound machine 1 via a bearing, the support disc 5 is coaxially welded to an output shaft of the first motor 19, a horizontal second motor 6 is welded to the support disc 5, a support arm 10 is pivotally connected to the center of the support disc 5, and an output shaft of the second motor 6 is coaxially welded to a pivot point at a bottom end of the support arm 10, thereby adjusting the rotation of the support arm 10. A bidirectional motor is horizontally welded to a middle portion of the support arm 10, a motor gear 7 is coaxially welded to each of output ends on two sides of the bidirectional motor, an extension arm 11 is pivotally connected to a top end of the support arm 10, an arm gear 8 is coaxially welded to each of two sides of a pivot point at a bottom end of the extension arm 11, and a transmission chain 9 is sleeved between the motor gear 7 and the corresponding arm gear 8, pulling the rotation of the extension arm 11. A plurality of electric push rods 16 are unidirectionally welded to a side wall of a top of the extension arm 11, a display 14 is ball-jointed to a top end of the extension arm 11, and each of the electric push rods 16 is ball-jointed to the display 14, thereby pushing the display 14 to change the angle. A camera 17 and a laser radar 18 are installed on the display 14, and a protective shell is fixedly sleeved on a periphery of each of the support disc 5, the support arm 10, and the extension arm 11. The first motor 19, the second motor 6, the bidirectional motor, the electric push rod 16, the display 14, the camera 17, and the laser radar 18 are all in signal connection with the controller 2.

A light shield 12 is pivotally connected to a top wall of the display 14, one end of a pull rope 15 is pivotally connected to a top wall of the light shield 12, the other end of the pull rope 15 is pivotally connected to one side of a bottom end of the extension arm 11, one side of a retractable light curtain 13 is bonded to each of two sides of the light shield 12, another side of the retractable light curtain 13 is bonded to a corresponding side of two sides of the display 14, and the light shield 12 and the retractable light curtains 13 are black.

The specific implementation process is as follows: In order to achieve facial tracking of the operator by the display 14 and always maintain the clearest observation angle of the display 14, it is first necessary to achieve automatic three-dimensional angle change of the display 14, where the structure supporting the display 14 consists of the support arm 10 and the extension arm 11 that are sequentially pivotally connected. The first motor 19 can rotate the support disc 5 to achieve overall radial rotation of the support structure and the display 14, the second motor 6 can drive the support arm 10 to rotate radially with the bottom pivot point as the center, and the bidirectional motor on the support arm 10 transmits power to the arm gear 8 and the extension arm 11 through the motor gear 7 and the transmission chain 9, which can drive the extension arm 11 to rotate radially with the pivot point between the extension arm 11 and the support arm 10 as the center. The electric push rods 16 on the side wall of the top of the extension arm 11 can push the angle transformation of the display 14 itself, thereby achieving changes in the radial axial angle and height of the display 14 at the top of the B-mode ultrasound machine 1, and laying the foundation for facial tracking display.

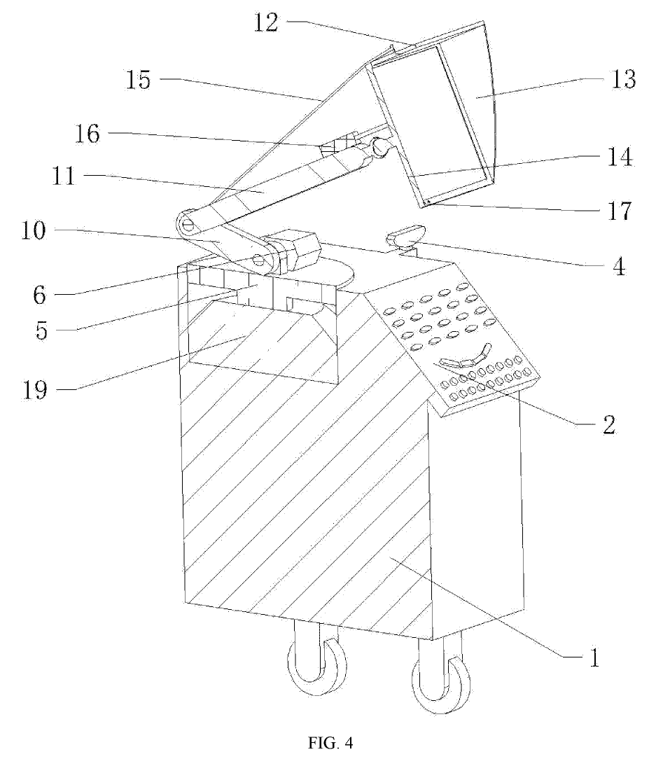

As shown in FIG. 4, since B-mode ultrasound displays are mostly black and white or monochrome, clear observation requires a high-contrast environment, such as a dark room. In this case, to avoid interference with the display effect of the display 14 by external light, the light shield 12 is pivotally connected to the display 14, but when the angle of the display 14 is adjusted, the light shield 12 may block the display content. Therefore, the pull rope 15 is added, pivotally connected between the extension arm 11 and the light shield 12. When the display 14 tilts downward, the light shield 12 is automatically pulled up to an elevation angle by the pull rope 15 to avoid blocking the display content, and the retractable light curtains 13 can completely shield interfering light from the sides of the display 14 under any elevation angle of the light shield 12, thereby enhancing display clarity.

### Embodiment 2

The difference from the above embodiment is that, as shown in FIGS. 1, 2, 3, and 4: a real-time audio receiving device is installed in the controller 2, one side of the B-mode ultrasound machine 1 is bonded to one end of a cable, the other end of the cable is clamped to a handheld ultrasound probe 4, a remote controller 3 is bonded to a tail of the ultrasound probe 4, and a plurality of buttons and knobs are installed on the remote controller 3, such that parameters of the B-mode ultrasound machine 1 can be remotely adjusted through the controller 2, where the real-time audio receiving device and the remote controller 3 are in signal connection with the controller 2.

The specific implementation process is as follows: During an ultrasonic detection process, an operator holds the ultrasound probe 4 to perform detection on a patient's abdomen. To help the operator to adjust parameters of the B-mode ultrasound machine 1 at any position, the remote controller 3 is installed on the handheld ultrasound probe 4 and can control changes in the angle of the display 14 and adjustments to parameters of the B-mode ultrasound machine 1, thereby facilitating remote operation by the operator without needing to move to the front of the B-mode ultrasound machine 1, improving work efficiency of ultrasonic detection, and reducing the labor of the operator.

The camera 17 captures images in front of the display 14, and the laser radar 18 obtains depth data of objects in front of the display 14, the depth data being combined with data of the images to form structured light data, and the real-time audio receiving device receives voice data from the operator. These data are the basis for achieving operator facial tracking display and non-contact control.

### Embodiment 3

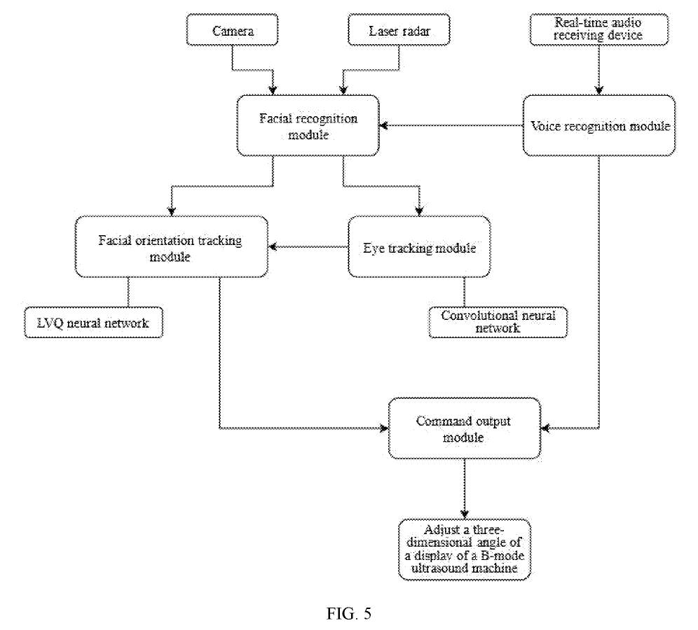

The difference from the above embodiments is that, as shown in FIG. 5: A facial recognition module is disposed in the controller 2, configured to combine image data of the camera 17 and depth data of the laser radar 18, to record facial features of an operator, and to identify a recorded operator.

A facial orientation tracking module is configured to combine the image data of the camera 17 and the depth data of the laser radar 18, to use a pre-trained LVQ neural network to recognize and track a face orientation, and to output three-dimensional angle data of the display 14. The LVQ neural network includes an input layer, a hidden layer, and an output layer, and is pre-trained using face images at different angles captured by the camera 17 and the depth data obtained by the laser radar 18.

An eye tracking module is configured to combine the image data of the camera 17 and the depth data of the laser radar 18, to use a pre-trained convolutional neural network to calculate an eye posture, to establish a gaze model, and to determine whether a line of sight of the operator is concentrated on a display screen.

The voice recognition module is configured to extract a voice feature of the operator and to identify voice text content.

A command output module is configured to, based on the three-dimensional angle data of the display 14, a line-of-sight determination result, and the voice text content, output an instruction to control a three-dimensional angle change of the display 14, to control starting or pausing of an angle change of the display 14, and to adjust parameters of the B-mode ultrasound machine 1.

The specific implementation process is as follows: Based on the above modules built into the controller 2, structured light data acquired by the camera 17 and the laser radar 18, and voice data acquired by the real-time audio receiving device can be used to control angle tracking of the display 14 and parameter adjustments of the B-mode ultrasound machine 1.

During the ultrasonic detection process, the operator turns on the B-mode ultrasound machine 1, and the facial recognition module identifies the recorded facial features of the operator to protect instrument safety, presets detection parameters, and starts detection. The operator holds the ultrasound probe to perform detection at different angles on the patient's abdomen. During this process, the operator may change position and posture for convenient holding of the ultrasound probe 4, even moving to the other side of the patient, and to obtain the most accurate detection results, the operator needs to constantly pay attention to the detection images displayed on the display 14. At this time, the apparatus can be set to enter the tracking mode automatically, or through voice recognition or control by the remote controller 3.

At this time, the operator is identified by the facial recognition module based on the structured light data formed by the data of images from the camera 17 and depth data from the laser radar 18. Based on the operator's facial orientation results calculated by LVQ neural network learning, the three-dimensional angle of the display 14 is adjusted to always face the operator's face directly. Based on learning and calculation of the operator's eye gaze posture by the convolutional neural network, the gaze line is extended, and it is determined whether the operator's line of sight is on the display 14. When the line of sight is on the display, the facial orientation tracking module always performs the tracking task to orient the display 14 towards the operator's face; when the line of sight leaves the display screen, the facial orientation tracking module pauses to save energy.

In some special cases, when the tracking starts, the operator may not be within the shooting range of the camera 17 and the laser radar 18, causing the facial recognition module to stop working. To resolve this, the operator may manipulate the display 14 to turn using the remote controller 3 or use special keywords to wake up voice mode, and then may control the display 14 to turn or adjust parameters of the B-mode ultrasound machine 1 through voice commands, such as waking up the voice mode with words "Xiao Chao, Xiao Chao". Then, the operator controls the rotation of the display 14 with a command "turn left 15 degrees". The combination of structured light recognition and tracking with voice control greatly enhances the adaptability of the apparatus in different scenarios, improves the practical application value of obtaining the best observation angle for ultrasonic detection operators, reduces the tediousness of repeatedly moving to adjust parameters of the B-mode ultrasound machine 1 in ultrasonic detection, increases the detection work efficiency of the operator, and indirectly improves detection accuracy.

It should be noted that, in this document, relational terms such as first and second are merely used to distinguish one entity or operation from another entity or operation, without necessarily requiring or implying any such actual relationship or order between these entities or operations. Moreover, the terms "include", "comprise", or any other variations thereof are intended to cover non-exclusive inclusion, so that a process, method, article, or device including a series of elements not only includes those elements, but also includes other elements not explicitly listed, or also includes elements inherent to such process, method, article, or device.

The above are only embodiments of the present invention, common knowledge such as specific structures and characteristics known in the scheme are not described too much here, and those of ordinary skill in the art know all ordinary technical knowledge in the technical field to which the invention belongs before the application date or priority date, can know all prior art in the field, and have the ability to apply conventional experimental means before that date. Those of ordinary skill in the art can, under the enlightenment given by this application, combine their own abilities to improve and implement this scheme. Some typical known structures or known methods should not become obstacles for those of ordinary skill in the art to implement this application. It should be pointed out that, for those skilled in the art, without departing from the structure of the present invention, several deformations and improvements can also be made, which should also be regarded as the protection scope of the present invention, and these will not affect the implementation effect of the present invention and the practicability of the patent. The protection scope required by this application should be based on the content of its claims, and the records such as specific implementations in the specification can be used to explain the content of the claims.

What is claimed is:

1. An ultrasound apparatus integrating diagnosis and therapy, comprising a B-mode ultrasound machine, wherein a controller is disposed on the B-mode ultrasound machine, a first motor oriented vertically upward is fixedly connected to a top of the B-mode ultrasound machine, a support disc is rotatably connected to a top wall of the B-mode ultrasound machine, the support disc is coaxially and fixedly connected to an output shaft of the first motor, a horizontal second motor is fixedly connected to the support disc, a support arm is pivotally connected to a center of the support disc, an output shaft of the second motor is coaxially and fixedly connected to a pivot point at a bottom end of the support arm, a bidirectional motor is horizontally and fixedly connected to a middle portion of the support arm, a motor gear is coaxially and fixedly connected to each of output ends on two sides of the bidirectional motor, an extension arm is pivotally connected to a top end of the support arm, an arm gear is coaxially and fixedly connected to each of two sides of a pivot point at a bottom end of the extension arm, a transmission chain is sleeved between the motor gear and the corresponding arm gear, a plurality of electric push rods are unidirectionally and fixedly connected to a side wall of a top of the extension arm, a display is ball-jointed to a top end of the extension arm, each of the electric push rods is ball-jointed to the display, and a camera and a laser radar are fixedly connected to the display, and
the first motor, the second motor, the bidirectional motor, the electric push rod, the display, the camera, and the laser radar are all in signal connection with the controller.

2. The ultrasound apparatus integrating diagnosis and therapy according to claim 1, wherein one side of the B-mode ultrasound machine is fixedly connected to one end of a cable, the other end of the cable is fixedly connected to a handheld ultrasound probe, a remote controller is fixedly connected to a tail of the ultrasound probe, a plurality of buttons and knobs are disposed on the remote controller, and the remote controller is in signal connection with the controller.

3. The ultrasound apparatus integrating diagnosis and therapy according to claim 1, wherein a real-time audio receiving device is disposed in the controller, and the real-time audio receiving device is in signal connection with the controller.

4. The ultrasound apparatus integrating diagnosis and therapy according to claim 1, wherein a plurality of casters are fixedly connected to a bottom wall of the B-mode ultrasound machine.

5. The ultrasound apparatus integrating diagnosis and therapy according to claim 1, wherein a light shield is pivotally connected to a top wall of the display, one end of a pull rope is pivotally connected to a top wall of the light shield, and the other end of the pull rope is pivotally connected to one side of the bottom end of the extension arm.

6. The ultrasound apparatus integrating diagnosis and therapy according to claim 5, wherein each of two sides of the light shield is fixedly connected to one end of a retractable light curtain, and the other end of the retractable light curtain is fixedly connected to a corresponding side of two sides of the display.

7. The ultrasound apparatus integrating diagnosis and therapy according to claim 6, wherein the light shield and the retractable light curtains are black.

8. The ultrasound apparatus integrating diagnosis and therapy according to claim 1, wherein a protective shell is fixedly sleeved on a periphery of each of the support disc, the support arm, and the extension arm.

9. The ultrasound apparatus integrating diagnosis and therapy according to claim 1, wherein a facial recognition module, a facial orientation tracking module, an eye tracking module, a voice recognition module, and a command output module are disposed in the controller, wherein
the facial recognition module is configured to combine image data of the camera and depth data of the laser radar to record facial features of an operator and to identify a recorded operator;
the facial orientation tracking module is configured to combine the image data of the camera and the depth data of the laser radar, to use a pre-trained LVQ neural network to recognize and track a face orientation, and to output three-dimensional angle data of the display;
the eye tracking module is configured to combine the image data of the camera and the depth data of the laser radar, to use a pre-trained convolutional neural network to calculate an eye posture, to establish a gaze model, and to determine whether a line of sight of the operator is concentrated on a display screen;
the voice recognition module is configured to extract a voice feature of the operator and to identify voice text content; and
the command output module is configured to, based on the three-dimensional angle data of the display, a line-of-sight determination result, and the voice text content, output an instruction to control a three-dimensional angle change of the display, to control starting or pausing of an angle change of the display, and to adjust parameters of the B-mode ultrasound machine.

10. The ultrasound apparatus integrating diagnosis and therapy according to claim 9, wherein the LVQ neural network in the facial orientation tracking module comprises an input layer, a hidden layer, and an output layer, and is pre-trained using face images at different angles captured by the camera and the depth data obtained by the laser radar.
